(19) United States
(12) Patent Application Publication
(10) Pub. No.: US 2010/0190975 A1
(43) Pub. Date: Jul. 29, 2010

(54) Method for purification of chlorinated sucrose derivatives by solvent extraction

(75) Inventors: Rakesh Ratnam, Maharashtra (IN); S. P. Raviraj, Mumbai (IN); Sundeep Aurora, Mumbai (IN)

Correspondence Address: THE NATH LAW GROUP, 112 South West Street, Alexandria, VA, 22314, US

(73) Assignee: PHARMED MEDICARE PRIVATE LIMITED, Mumbai, Maharashtra (IN)

(21) Appl. No.: 11/921,183

(22) PCT Filed: May 26, 2006
(86) PCT No.: PCT/IN2006/000181

§ 371 Date: Nov. 28, 2007

(30) Foreign Application Priority Data
Jun. 1, 2005 (IN) 654/MUM/2005

(51) Int. Cl. C07H 1/06

(52) U.S. Cl. 536/127

(57) ABSTRACT

A process is described for extractive isolation and purification of Trichlorogalactosucrose from impurities from a composition substantially free from N-N Dimethylformamide (DMF) comprising a first extractive separation by adjusting aqueous to a mixture of organic solvents in the composition to a certain ratio of aqueous to organic phase, extracting the aqueous layer of this step by a single organic solvent, followed by saturation of the aqueous layer by salt and ultimately extracting TGS in the organic layer.

### TECHNICAL FIELD

The present invention relates to a process and a novel strategy for isolation and purification of 1′-6′-Dichloro-1′-6′-DIDEOXY-β-Fructofuranasyl-4-chloro-4-deoxy-galactopyranoside (TGS) and other chlorinated sucrose derivatives from a reaction mixture by solvent extraction.

### BACKGROUND OF INVENTION

Chlorinated sucrose preparation is a challenging process due to the need of chlorination in selective less reactive positions in sucrose molecule in competition with more reactive positions. Generally, this objective is achieved by a procedure which involves essentially protecting the hydroxy group in the pyranose ring of sugar molecule by using various protecting agents alky/aryl anhydride, acid chlorides, orthoesters etc., and the protected sucrose is then chlorinated in the desired positions (1′-6′ &, 4) to give the acetyl derivative of the product, which is then deacylated to give the desired product 1′-6-Dichloro-1-6-DIDEOXY-β-Fructofuranasyl-4-chloro-4-deoxy-galactopyranoside i.e. 4,1′, 6′ trichlorogalactosucrose i.e. TGS.

Strategies of prior art methods of production of TGS are based on following: Sucrose-6-acetate is chlorinated by Vilsmeier Haack reagent to form TGS-6-acetate. After chlorination, the deacetylation of TGS-6-Acetate to TGS is carried out in the reaction mixture itself. The process is described in greater details in various patents and in patent applications including Ratnam et al (2005) in WO2005090374 and Ratnam et al (2005) in WO2005090376. As an inevitable part of above synthesis strategy, various other chloro substitution products are also produced in varying amounts. Isolation of TGS from the other organic impurities by liquid-liquid extraction is usually a daunting task due to affinity of the product to hydrophilic as well as hydrophobic solvents.

None of above prior art patents however, cover the process of this invention wherein the strategy of solvent extraction is governed by controlling the ratio of aqueous phase and organic phase in the first step (the organic phase being contributed here by a mixture of organic solvents) which results in removal of a group of impurities in the organic layer, followed by extraction of the aqueous layer by a single organic solvent to extract next group of impurities in the organic layer, followed by salt saturation of the aqueous layer and extraction of TGS contained in it by an organic solvent which selectively extracts TGS in it leaving behind rest of the impurities in the aqueous layer.

### PRIOR ART

Extractive purification by solvents has been extensively covered by Catani et al (2003) in US 20030171574/WO0376453. It involves six independent claims which describe various combinations of solvent extractive step, each combination involving at least two additional solvents extracting the original compositions containing TGS and impurities.

Mufti et al (1983) in U.S. Pat. No. 4,380,476 has reported extraction of an aqueous solution with dichloromethane (to remove tetrachloro derivatives) and then with ethyl acetate.

### SUMMARY OF INVENTION

This invention embodies a process for isolation of chlorinated sucrose derivatives from a solution by solvent extraction by controlling ratio of aqueous phase to organic solvent (v/v). In the preferred embodiment, this solution is results from aqueous extraction of mixture of solidmass derived from dried process flow reaction mixture after chlorination of sucrose-6-acetate by Vilsmeier reagent. In an other embodiment of this invention, the improved solvent extraction process can also be applied to any other solution of TGS, aqueous or organic, derived from any other process of production, wherein TGS needs to be isolated and extracted from other inorganic and/or organic impurities. All adaptations of the scheme of solvent extraction strategy described herein and covered in the claims are covered as embodiments of this invention.

One embodiment of the strategy of solvent extraction of this process involves preferential extraction of chlorinated sucrose derivatives in relatively pure form using differential solubilities of various closely related compounds in different ratios of aqueous to organic phase where the organic phase is provided by one organic solvent or a mixture of organic solvents. This process surprisingly accomplishes isolation of TGS with high purity levels at far greater ease from closely related impurities in a reaction mixture derived from deacylation of chlorinated mixture which may contain one or more of, usually more of, (a) 4, 6, 1′, 6′-tetrachlorogalactosucrose, (b) 4,1′, 4′, 6′-tetrachlorogalactosucrose, (c) 6-acetyl, 4, 1′, 6′-trichlorogalactosucrose, (d) 4,1′, 6′-trichlorogalactosucrose, (e) 1′, 6′-dichlorosucrose and (f) 4, 1′-dichlorosucrose with a solvent extraction strategy involving, serially, following steps:
a. Adjusting a volume/volume ratio of aqueous phase: Organic phase to 1:0.25 to 1:0.3 of a substantially N,N-dimethylformamide (DMF) free solution containing TGS and impurities, wherein the organic layer selectively extracts the compounds ‘a’ & ‘b’. The said solution containing TGS is obtained in substantially DMF free state either by dissolving in water a solid mixture of the chemicals to be separated, or by azeotropic distillation in rising film evaporators or falling film evaporators, or by obtaining an organic solvent extract of a liquid reaction mixture containing TGS in a relatively water immiscible solvent, including but not limited to ethyl acetate, which is washed by saturated solution of sodium chloride in water. The organic layer could be a single organic solvent or a mixture of two or more solvents.
b. Further extraction of the aqueous layer with a single water immiscible or sparingly miscible solvent such as methylene dichloride, ethyl acetate, ethylene dichloride, methyl ethyl ketone, etc. wherein the volume/volume ratio of the aqueous phase to organic phase is adjusted to 1:0.3 to 1:0.35 of wherein organic layer selectively extracts the compound ‘c’. If esters of a tetrachlorosucrose and a dichlorosucrose, which were not detected and hence not isolated and characterized by us, also get formed and become a part of the reaction mixture, they shall also be selectively removed in this step along with ‘C’.
c. Further, saturation of the aqueous layer with a salt including but not limited to sodium chloride, preferably adjusting the pH to neutral and extraction of the aqueous layer with 1: 1.2 to 1.5 times v/v of an organic solvent sparingly miscible or immiscible in water such as ethyl acetate, Methyl ethyl ketone, butyl acetate, etc. wherein the ‘d’ i.e. TGS is selectively extracted into the organic solvent and compounds left out in the aqueous layer are ‘e’ & ‘f’.

There could be other impurities too which were not detected by us, but which were nevertheless removed in one or the other layers discarded by us during the scheme of solvent extraction describe here. In a variation of above extraction scheme, a process step may be omitted if a constituent relevant to the process step is not present in the solution to be extracted.

### DETAILED DESCRIPTION OF THE INVENTION

Throughout this specification including claims, a mention in singular shall also include plural and equivalents or homologous. For example, mention of “an organic solvent” shall include one or more of all known organic solvents; or mention of “a process of production of TGS” includes one or more of all processes which can be described as a process for production of TGS.

The new strategy of solvent extraction originated from a surprising finding that proportions, by volume, of aqueous to organic phases in a solvent extraction system plays a very significant role in allowing selective extraction/isolation of closely related chemical compounds. This finding was explored to develop a process from this revolutionary concept of solvent extraction for chlorinated sucrose derivatives in particular and for industrially useful compounds in general other than chlorinated sugars and their derivatives using the principle of varying proportions of aqueous and organic phase and their various combinations. The said solution of TGS to which process of this invention is applicable could also be a derivative of a process of production of TGS not involving chlorination reaction and involving only one or none of a chlorinated sucrose other than TGS. TGS isolated in this way from any of its solutions could be further taken up for crystallization for its eventual conversion into a solid form.

In a preferred embodiment, which shows working of the invention, reaction mixture derived from chlorination of sucrose-6-acetate by the Vilsmeier-Haack reaction is neutralized and is taken for Agitated Thin Film Drier is (ATFD) drying as described by Ratnam et al (2005a) in WO2005090374 and Ratnam et al (2005b) WO2005090376. This reaction mixture may be taken for drying before deacylation, after deacylation, before removal of one or more of the constituents of the mixture such as solvents including Dimethylformamide (DMF) by one or more methods of separation.

In another embodiment, the reaction mixture can be directly extracted into water immiscible or sparingly water miscible solvents such as ethyl acetate, etc and then concentrating the solvent extract and removal of DMF to a large extent by washing the organic extract using saturated sodium chloride solution in water. Further, after the removal of DMF, the isolation of chlorinated sucrose derivatives is proceeded.

In other embodiments and situations not derived from reaction mixtures, solutions needing isolation of one or more chlorinated sucrose derivatives can also be involved which are derived from processes other than chlorination and may include forms of chlorinated sucrose taken initially in .amorphous or crystalline form or in a liquid or syrupy form.

The chlorinated sucrose derivatives present in the solids obtained after ATFD drying in the reaction mixtures of production of chlorinated sucrose in prior art methods were isolated and characterized. They were found to be as follows:
a) 4, 6, 1′, 6′-tetrachlorogalactosucrose. Molecular Formula=C₁₂ H₁₈ Cl₄ O₇; Molecular Weight 416.05.
b) 4,1′, 4′, 6′-tetrachlorogalactosucrose. Molecular Formula=C₁₂ H₁₈ Cl₄ O₇; Molecular Weight 416.05.
c) 6-acetyl, 4, 1′, 6′-trichlorogalactosucrose. The molecular formula=C₁₂H₂₁Cl₃O₉; Molecular Weight=439.6
d) 4, 1′, 6′-trichlorogalactosucrose: The molecular formula=C₁₂H₁₉Cl₃O₈; Molecular Weight=397.64.
e) 1′, 6′-dichlorosucrose. Molecular Formula=C₁₂H₂₀Cl₂O₉ ; molecular weight=379.19;
f) 4, 1′-dichlorosucrose. molecular formula=C₁₂H₂₀Cl₂O₉; Molecular Weight=379.19

Although we did not detect other impurities, their occurrence is not ruled out, however, in the scheme of solvent extraction described here, TGS that gets isolated is always pure and impurities get discarded in one or the other layer that is discarded.

Before subjecting the reaction mixture to ATFD drying or any other method of drying including spray drying, freeze drying, drying by supercritical extraction, evaporative drying etc., the DMF load can be reduced by adopting various novel strategies not used in prior art, some of which are as follows:

The load of the tertiary amide such as DMF in the neutralized mass for spray drying and ATFD can be reduced by subjecting the neutralized mass to concentration in specialized liquid-liquid extraction equipments such as the Rising Film Evaporator (RFE), Falling Film Evaporator (FFE), etc.

The neutralized reaction mass when subjected to RFE, part of the tertiary amide (DMF) along with water as an azeotrope is distilled off. The volume that was distilled off is reconstituted with water back in the neutralized mass. This process could be repeated a number of times to remove the tertiary amide (DMF) in each cycle. And at the end of each cycle, the loss in volume is reconstituted with water.

Then the neutralized mass which predominantly consists of water with reduced amount of the tertiary amide is subjected to drying process. By this method, the loading of the tertiary amide such as DMF in the reaction mass is reduced.

The reaction mass can also be directly extracted into water immiscible solvent such as ethyl acetate, concentrated and then washed with saturated sodium chloride (brine) solution for removal of DMF and then taken directly for isolation of chlorinated sucrose derivatives.

The solids obtained from the Agitated Thin Film Drier (ATFD) or liquid process flow compositions, containing TGS and impurities, after brine washings contained both inorganic salts and organic compounds which included the chlorinated sucrose derivatives. The said solids, depending on any variation of reactants or process steps, may contain a different composition of chemicals including presence of either only inorganic or only organic compounds too. The solids were dissolved in 1.5 times to 5 times by weight in water, more preferably 1.8 to 2.5 times. The suspended solids were filtered using an appropriate filter aid. The pH of the clear filtrate obtained was adjusted to neutral and was extracted with proportionate amount of mixture of solvents including Ethyl acetate, butyl acetate, methylene dichloride, ethylene dichloride, toluene, cyclohexane, etc in combination. The ratio of the aqueous to the organic solvent mixture was adjusted to 1:0.25 to 1:0.55 v/v preferably 1:0.4 to 1:0.45 v/v respectively The extraction was repeated two to four times, more preferably two times. The extracted organic layer was analyzed and was found to contain the compounds ‘a’ & ‘b’.

The aqueous layer was further extracted proportionate amount of a single water immiscible or sparingly miscible solvent such as methylene dichloride, ethyl acetate, ethylene dichloride, methyl ethyl ketone, etc. The ratio of the aqueous to organic solvent was adjusted to 1:0.3 to 1:0.35 v/v respectively. The extraction was repeated two to four times more preferably two times.

The extracted organic layer was analyzed and was found to contain the compound ‘c’.

The aqueous layer was then saturated with sodium chloride and the pH was once again adjusted to neutral. The aqueous layer was then extracted with 1:1.2 to 1.5 times v/v of organic solvent such as ethyl acetate, Methyl ethyl ketone, butyl acetate, etc., twice. The compound extracted in the organic solvent was found to be ‘d’. The compounds left out in the aqueous layer were found to be ‘e’ & ‘f’.

If some amount of compound ‘d’ left over in aqueous, the aqueous layer pH was re-adjusted to neutral and further saturation with sodium chloride was ascertained. The aqueous layer was further extracted with 0.6-0.8 times v/v of organic solvent such as ethyl acetate, butyl acetate, etc.

The organic layers containing compound ‘d’ that was TGS, which was pooled, charcoalized and concentrated under vacuum at 30-50° C. preferably at 40-45° C. temperature. During the concentration, the pH of the solution was maintained neutral using sodium carbonate. The organic solvent was evaporated off completely leaving behind a syrup containing some amount of water. The water was removed completely by addition of a suitable azeotrope such as cyclohexane, toluene, heptane, etc.

A thick syrupy mass was obtained to which 1:0.5 to 0.8 times v/v of ethyl acetate was added and a seeding of 2% of TGS was added and kept at room temperature for 24 hrs to 50 hrs for crystallization. The crystals were then filtered, washed with methylene chloride and analyzed by HPLC.

The other organic layers obtained at various stages explained were also concentrated and analyzed. From the purified solution at the end of above solvent extraction scheme, TGS can be isolated in solid form by using crystallization. However, any other treatment can be given to such a solution of pure TGS, which includes but is not limited to adsorption on a suitable adsorbent, thorough washing to make it free from other constituents of the solution subjected to adsorption on the adsorbent, elution of TGS into other aqueous or organic solvent and separation into a solid form by a suitable process including drying by various methods comprising ATFD drying, freeze drying, spray drying and the like.

An adaptation or modification such as only partially following the scheme of extractive purification only to a certain step and then switching over to another method of purification is also included within the scope of this invention. Thus, either after solvent extractive removal of tetrachloro impurities, or after solvent extractive removal of TGS-acetate, the aqueous layer can be subjected to isolation of TGS by one or more of a column chromatographic isolation method including but not limited to column chromatography on silanized silica gel and the like.

At the same time a modification or adaptation of scheme of solvent extractive purification may be applied from any advanced step, omitting earlier steps, depending on the aim of purification and as long as the aim of purification is achieved; and such adaptations or modifications are also involved within the scope of this invention. For example, if only isolation of TGS is aimed at from a neutralized chlorination reaction mixture substantially free from DMF, it may be possible to achieve it by extraction of the aqueous layer with a single water immiscible or sparingly miscible solvent such as methylene dichloride, ethyl acetate, ethylene dichloride, methyl ethyl ketone, and the like wherein the ratio of the aqueous phase to organic solvent is adjusted to 1:0.3 to 1:0.35 v/v respectively and then going to saturation of the aqueous layer with sodium chloride and extracting it with a single water immiscible or sparingly miscible solvent such as methylene dichloride, ethyl acetate, ethylene dichloride, methyl ethyl ketone, It may be a possible variation that extraction of TGS from saturated sodium chloride solution is done by a combination of organic solvents. Thus, many more variations and adaptations of the invention claimed in this specification are possible and all are included within the scope of the claims of this invention.

Examples are described below which serve the purpose of illustrating how the invention works for isolation of TGS or other chlorinated sucrose derivatives in a reaction mixture/a process stream in the manufacture of a chlorinated sucrose including TGS, from other components without putting any limitations on the composition of a reaction mixture/a process stream taken, or on source of a reaction mixture/a process stream taken for isolation and separation. Any application of this invention analogous to the claims and obvious to a person skilled in the art is included within the scope of this invention.

### EXAMPLES

### Example 1

ATFD solids mass, 50 kg, was dissolved in 75 L of de-mineralized water and was centrifuged to remove the insoluble solids. The filter aid used was Hyflo super cell. The clear filtrate obtained along with washings was 80 L was contacted with 36 L of organic solvent mixture containing 80% ethyl acetate and 20% of cyclohexane. The layers were separated and the aqueous layer was again contacted with a fresh 36 L of the same organic solvent mixture. The layers were again separated.

The separated layers were analyzed by Thin Layer Chromatography and the movement of certain compounds from aqueous to organic layers were recorded. The TLC system mobile phase used was 8:6:1 (Ethyl acetate: Acetone: Water). The aqueous layer was then contacted with 24 L of ethyl acetate twice and layers separated. The separated layers were again recorded by TLC.

The aqueous layer was then saturated with sodium chloride (17.6 kg). The pH of the aqueous layer was adjusted to neutral using 20% sodium carbonate solution. The aqueous layer was then extracted with 95 L of ethyl acetate twice and the migration of TGS to the organic layer was checked by TLC. The aqueous layer was further saturated with 8.0 kg of sodium chloride and extracted with 40 L of ethyl acetate. The layers were separated and the aqueous layer was concentrated under vacuum at 55° C. and the sodium chloride crystals were filtered off. The final syrup free from inorganic salts was analyzed.

The organic layers were pooled together and 11.5 kg of charcoal was added and stirred for 45 min at room temperature. The solution was then filtered to remove charcoal and then concentrated under vacuum at 45° C. up to 15 L stage where ethyl acetate was completely removed. 25 L of cyclohexane was added and refluxed to remove water from the mass. A thick syrupy mass of about 4.0 kg was obtained to which about 2.5 L of ethyl acetate was added.

The product content was analyzed by HPLC in the syrupy mass and was found to be 62%. 50 g of TGS was added as seeding and the contents were kept for crystallization for 48 hr. The crystals were then filtered (1.2 kg) and washed with 2.0 L methylene dichloride and were dried. The mother liquor was concentrated and again seeded and kept for crystallization. A second crop of 800 g of crystals was obtained from the mother liquor. The product was blended and analyzed by HPLC and was found to be 99% pure.

### Example 2

2000 L of neutralized mass was subjected to ethyl acetate extraction in a liquid liquid extraction column. The volume/volume (v/v) ratio of ethyl acetate to neutralized mass for extraction was 3.5:1. The ethyl acetate extract thus separated was analyzed for TGS content and DMF content. It was seen that about 10% of the total DMF in the neutralized mass partitioned into the ethyl acetate extract. The TGS from the neutralized mass was completely extracted into ethyl acetate and the extract was subjected to distillation under vacuum. When the volume of the extract reached 750 L, the concentration was stopped and the water from extract separated out. This water was removed. 0.25% v/v of saturated sodium chloride solution was added to the organic concentrate and mixed thoroughly for 15 minutes and allowed to settle. The layers were separated. This addition of saturated sodium chloride solution, mixing and separation was repeated 8 to 10 times till the DMF content in the organic extract reached less than 1%. It was seen that the TGS leaching into the brine washing was not more than 3%.

After the DMF removal by brine washings, the organic extract was further concentrated to maximum, taken in water and deacetylated using sodium hydroxide. The deacetylated mass was about 80 L was taken for the to isolation of TGS.

This solution was contacted with 36 L of organic solvent mixture containing 80% ethyl acetate and 20% of cyclohexane. The layers were separated and the aqueous layer was again contacted with a fresh 36 L of the same organic solvent mixture. The layers were again separated.

The separated layers were analyzed by Thin Layer Chromatography and the movement of certain compounds from aqueous to organic layers were recorded. The TLC system mobile phase used was 8:6:1 (Ethyl acetate: Acetone: Water). The aqueous layer was then contacted with 24 L of ethyl acetate twice and layers separated. The separated layers were again recorded by TLC.

The aqueous layer was then saturated with sodium chloride (17.6 kg) The pH of the aqueous layer was adjusted to neutral using 20% sodium carbonate solution. The aqueous layer was then extracted with 95 L of ethyl acetate twice and the migration of 4,1′, 6′-trichlorogalactosucrose to the organic layer was checked by TLC. The aqueous layer was further saturated with 8.0 kg of sodium chloride and extracted with 40 L of ethyl acetate. The layers were separated and the aqueous layer was concentrated under vacuum at 55° C. and the sodium chloride crystals were filtered off. The final syrup free from inorganic salts was analyzed.

The organic layers were pooled together and 11.5 kg of charcoal was added and stirred for 45 min at room temperature. The solution was then filtered to remove charcoal and then concentrated under vacuum at 45° C. up to 15 L stage where ethyl acetate was completely removed. 25 L of cyclohexane was added and refluxed to remove water from the mass. A thick syrupy mass of about 4.0 kg was obtained to which about 2.5 L of ethyl acetate was added.

The product content was analyzed by HPLC in the syrupy mass and was found to be 62%. 50 g of 4,1′, 6′-trichlorogalactosucrose was added as seeding and the contents were kept for crystallization for 48 hr. The crystals were then filtered (1.2 kg) and washed with 2.0 L methylene dichloride and was dried. The mother liquor was concentrated and again seeded and kept for crystallization. A second crop of 800 g of crystals was obtained from the mother liquor. The product was blended and analyzed by HPLC and was found to be 96.7%.

Further work along above scope of plan of work is progressing and is being documented.

1. A process for isolation and purification of 1′-6′-Dichloro-1′-6′-DIDEOXY-β-Fructofuranasyl-4-chloro-4-deoxy-galactopyranoside (TGS) from a composition at least about 96 percent free from N-N Dimethylformamide (DMF), containing TGS and one or more of impurities comprising salts and derivatives of one or more of a chlorinated sucrose comprising following steps:
i. adding to the above said composition a mixture of organic solvent, and if required water, to achieve ratio of aqueous to organic phase such that substantially selective extraction of 4, 6, 1′, 6′-tetrachlorogalactosucrose, 4,1′, 4′, 6′-tetrachlorogalactosucrose and impurities equally or more hydrophobic thereof are extracted in the organic layer; including a ratio of aqueous phase to organic phase as 1:0.25 to 1:0.55 v/v, preferably from 1: 0.4 to 1: 0.45 v/v, wherein the said mixture of organic solvents is preferably of ethyl acetate and cyclohexane taken further preferably at a ratio of 80% to 20% respectively,
ii. subjecting the aqueous layer of step (i.) to extraction with a single water immiscible or sparingly miscible solvent comprising one or more of methylene dichloride, ethyl acetate, ethylene dichloride, methyl ethyl ketone, etc. wherein the ratio of the aqueous to organic solvent is adjusted such that substantially selective extraction of ester derivatives of tetrachlorogalactosucrose, trichlorogalactosucrose and dichlorosucroses and impurities equally or more hydrophobic thereof are extracted in the organic layer, including a ratio of 1:0.3-1:0.35 of organic solvent v/v respectively,
iii. saturating the aqueous layer of step (ii.) with a salt including but not limited to sodium chloride, preferably adjusting the pH to neutral and extraction of the aqueous layer with 1: 1.2 to 1.5 times v/v of an organic solvent sparingly miscible or immiscible in water such as ethyl acetate, Methyl ethyl ketone, butyl acetate and the like, to achieve extraction of TGS in the organic layer and
iv. recovering TGS from the organic phase of step iii.) by one or more of a method of isolating a solid from a liquid comprising crystallization, column chromatography and the like.

2. A process of claim 1 wherein:
i. the said composition comprises a process stream in a process of production of TGS, or
ii. the said composition is a solution containing TGS obtained by dissolving in water a solid mixture of TGS and impurities followed optionally by filtration to remove un dissolved solids, where the said solid mixture may be a result of drying of a process stream in a process of production of TGS.

3. A process of claim 1 wherein:
i. the said salts include one or more of a salt of one or more of an alkali metal, an alkaline earth metal and the like, and
ii. the said derivative of a chlorinated sucrose is one or more of (a) 4, 6, 1′, 6′-tetrachlorogalactosucrose, (b) 4,1′, 4′, 6′-tetrachlorogalactosucrose, (c) ester derivatives of tetrachlorogalactosucrose, trichlorogalactosucrose and dichlorosucroses (d) 4,1′, 6′-trichlorogalactosucrose, (e) 1′, 6′-dichlorosucrose and (f) 4, 1′-dichlorosucrose, further

4. A process of claim 1 wherein the said composition is obtained substantially free from DMF by one or more of a method of DMF removal, excluding steam stripping, comprising:
i. azeotropic distillation in rising film evaporators or falling film evaporators, or
ii. extracting the said composition in a relatively water immiscible solvent and washing the same with a saturated solution of sodium chloride in water, the said relatively water immiscible solvent is a single organic solvent or a mixture of two or more solvents comprising ethyl acetate, Methyl ethyl ketone, butyl acetate and the like.

5. A process of claim 1 wherein step (i.) or step (ii) or both the steps of claim 1 are omitted in absence of impurities comprising terta-chloro sucrose, or sucrose-6-acetate or both and the like.

6. A process of claim 1 wherein step (1) of claim 1 is omitted and only step (ii) and (iii) are performed.

7. A process of claim 1 wherein step (ii) is omitted since sucrose-6-acylate is not present in the composition.
